(19) United States
(12) Patent Application Publication
(10) Pub. No.: US 2020/0146558 A1
(43) Pub. Date: May 14, 2020

(54) Apparatus, code, methods, and systems for providing unexpected rewards for a measured change of a user

(71) Applicants: Christopher Jones, Burlington, VT (US); Ted James, Dover, MA (US); John N. Evans, South Burlington, VT (US); Asim Zia, Shelbourne, VT (US); Arthur Brassert, Caux (FR)

(72) Inventors: Christopher Jones, Burlington, VT (US); Ted James, Dover, MA (US); John N. Evans, South Burlington, VT (US); Asim Zia, Shelbourne, VT (US); Arthur Brassert, Caux (FR)

(21) Appl. No.: 16/522,640

(22) Filed: Jul. 25, 2019

Related U.S. Application Data
(60) Provisional application No. 62/703,282, filed on Jul. 25, 2018.

(51) Int. Cl. A61B 5/0205; A61B 5/00; G06Q 30/02

(52) U.S. Cl. CPC A61B 5/0205; A61B 5/0022; G06Q 30/0211; A61B 5/6824; A61B 5/7275

(57) ABSTRACT

Code configured receiving, over the communications network from information sources associated with the user baseline data storing the baseline data in the attached database. A first algorithm is selected from algorithms stored in an attached database. The first algorithm is for facilitating the measured change of a user based upon the baseline data received. A performance indicator for facilitating the measured change is determined. Next, the code is configured for receiving from one of the information sources a first set of performance indicator user data, Next, the code determines based upon the performance indicator user data received if the performance indicator for the measured change was satisfied. Next, the code provides to the remote computing an unexpected reward message or the loss aversion message based upon if the performance indicator for the measured change was satisfied. The steps are repeated until the measured change is satisfied.

### CROSS-REFERENCE TO RELATED APPLICATIONS

This application claims priority to provisional application No. 62/703,282 filed on Jul. 25, 2019, which is incorporated by reference herein in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The present invention relates to the field of motivation and providing rewards.

### BACKGROUND

There is growing evidence that avoidance of weight gain may reduce the risk of certain cancers, especially post-menopausal breast cancer and aggressive variants of prostate cancer. In addition to benefits in cancer risk reduction, weight maintenance lowers the risk of other chronic diseases like heart disease and diabetes. Prior research has endeavored to identify approaches that encourage long-term increases in healthy behavior such as the consumption of fruits and vegetables. Baranowski et al. (2002) designed an intervention that awarded a special badge to participants who adopted healthier eating habits, showing that participants adopted a diet higher in fruit and low-fat vegetables immediately after the intervention; however, the diet was not maintained six months later. This study showed that behavior change is possible using even non-monetary incentives but underscores the need for alternative strategies to sustain long-term changes.

In addition to the U.S. population becoming progressively obese, the present healthcare system has swelled in terms of national spending to the point where it can no longer sustain itself. In response, the healthcare system is experiencing a monumental shift from volume to value. While it remains unclear as to how costs are going to be contained, one thing is certain: the diseases of today and the foreseeable future will continue to swell in the direction of chronic conditions that have unhealthy behavioral root causes. Health care delivery systems are going to need to respond to these behaviors in a more efficient manner, in order to ensure patients adopt a greater locus of accountability for the many complex aspects of health that can be modified.

To date, incentive-based research has largely taken the form of randomized controlled trials where patients are seen in weekly or monthly increments and rewarded using grant-funded cash incentives (e.g., $50 bills) contingent upon changes in behavior, like smoking cessation (Halpern et al. 2015) or kg of weight loss. One of the challenges is that this approach uses a “one size fits all” approach to incentivizing; when those studies are over, the money runs out and patients tend to relapse.

Behavioral interventions hold much promise for effectively changing physical activity behavior among cancer survivors. However, more research is needed to identify the best ways of supporting survivors to make and maintain these lifestyle changes. Tailored interventions for cancer survivors and at-risk individuals may be more effective when accounting for factors associated with health-promotion engagement. Identifying effective ways of assisting cancer survivors and at-risk individuals to adopt and maintain healthy lifestyles is important for enhancing the well-being and health outcomes of this group. There is a pressing need to direct more attention to this issue to inform the development of interventions that maintain healthy weight in order to avoid the downstream risk of chronic disease. While others have demonstrated that incentives work to motivate healthy behavioral change, traditional research programs that use incentives cost millions of dollars to implement and study.

As a result, there exists a need for improvements over the prior art and more particularly for a more efficient way of motivating people to change behavioral habits.

### SUMMARY

A system and method for providing unexpected rewards for a measured change of a user is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a computer readable medium storing computer readable program code which when executed on a server is configured for providing unexpected rewards for a measured change of a user is discloses. The code is configured for: a. providing, over a communications network, to a remote computing device a graphical user interface configured for receiving a plurality of user data and storing, in the attached database, the user data in a plurality of user records; b. receiving, over the communications network, from a plurality of information sources associated with the user, a plurality of baseline data for the user and storing the baseline data in the attached database; c. selecting from a plurality of algorithms stored in an attached database, a first algorithm for facilitating the measured change of a user based upon the baseline data received; d. determining, based upon the baseline data and the first algorithm, at least one performance indicator for facilitating the measured change, and storing the performance indicator in the corresponding user record; e. receiving, over the communications network, from at least one of the plurality of information sources associated a first set of performance indicator user data; f. determining based upon the first set of performance indicator user data received if the performance indicator for the measured change was satisfied; g. providing, over the communications network, to the remote computing at least one of an unexpected reward message, and the loss aversion message based upon if the performance indicator for the measured change was satisfied; and, h. repeating steps c-g until the measured change is satisfied.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is a diagram of an operating environment that supports apparatus, code, methods, and systems for providing unexpected rewards for a measured change of a user, according to an example embodiment;

FIG. 2 is a diagram showing the data flow of the apparatus, code, methods, and systems for providing unexpected rewards for a measured change of a user, according to an example embodiment;

FIG. 3 is a diagram showing a first process flow of the apparatus, code, methods, and systems for providing unexpected rewards for a measured change of a user, according to an example embodiment;

FIG. 4 is a diagram showing a second process flow of the apparatus, code, methods, and systems for providing unexpected rewards for a measured change of a user, according to an example embodiment;

FIG. 5 is a diagram showing a third process flow of the apparatus, code, methods, and systems for providing unexpected rewards for a measured change of a user, according to an example embodiment;

FIG. 6A is a diagram illustrating a first graphical displays used with the apparatus, code, methods, and systems for providing unexpected rewards for a measured change of a user, according to an example embodiment:

FIG. 6B is a diagram illustrating a second graphical display used with the apparatus, code, methods, and systems for providing unexpected rewards for a measured change of a user, according to an example embodiment; and,

FIG. 7 is a block diagram of a computing device system including an example computing device and other computing devices.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering, or adding additional stages or components to the disclosed methods and devices. Accordingly, the following detailed description does not limit the disclosed embodiments. Instead, the proper scope of the disclosed embodiments is defined by the appended claims.

The disclosed embodiments improve upon the problems with the prior art by providing a system that combines knowledge across research fields: clinical medicine, behavioral psychology, and health economics, towards an approach that incentivizes the prevention of weight gain as well as other positive changes using expected rewards. The positive changes may include improved blood glucoses; blood pressures, BMIs; body weight; calories consumed; calories consumed; heart rate; step count; total sleep; mindfulness; reproductive health; basal body temperature; sexual activities; menstrual flow; intermenstrual bleedings; and, ovulation tests, awake time, deep/light/REM sleep, distance, duration, a sleep pattern, a fat percentage, a muscle mass change, a change in reading habits, and cybergenic etc. The disclosed embodiments improve over the prior art by providing apparatus, code, methods, and systems that apply algorithms that receive information and data from a plurality of information sources and by providing unexpected reward messages and loss aversion messages to cause desired and undesired positive changes. The disclosed embodiments also improve over the prior art by providing algorithms that determine the efficacy of and adjust the unexpected reward messages and the loss aversion messages so that the user achieves a measured change. The disclosed embodiments also improve over the prior art by adjusting the predetermined amount of times for sending inquiry messages to the user such that user response messages are received within at least in the same amount of or less than the amount of time than the previous user message was received.

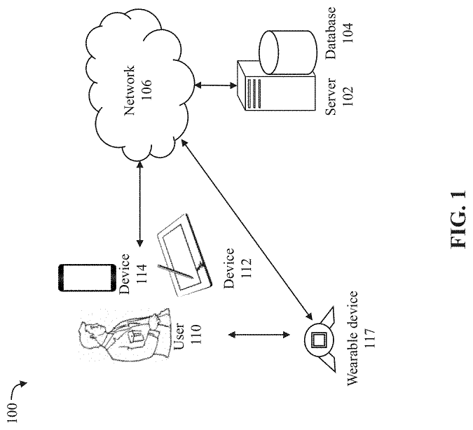

As a preliminary matter, Applicant hereby incorporates herein the Appendix filed with the specification. Referring now to the Figures, FIG. 1 is a diagram of an operating environment 100 that supports apparatus, code, methods, and systems, coupled to communication network 106, for providing unexpected rewards for a measured change of a user, according to an example embodiment. In other embodiments, the apparatus, code, methods, and systems are configured to operate on a single operating device without the need for communications networks. The environment 100 may comprises devices 112, 114, wearable device 117 and server 102, all of which may communicate with server 102 via a communications network 106. Devices 112, 114, wearable device 117 and server 102 may comprise any computing device, such as work stations, integrated circuits, printed circuit boards, processors, ASICs, PCBs, cellular telephones, smart phones, tablet computers, desktop computers, laptops, and game consoles, for example. Additionally, the wearable device 117 may communicate via radio frequency or vendor application. The wearable device may include wearable technology, fashionable technology, wearable devices, tech togs, or fashion electronics such as clothing and accessories incorporating computer and advanced electronic technologies.

Devices 112, 114, wearable device 117 and server 102 may be connected either wirelessly or in a wired or fiber optic form to the communications network 106. Communications network 106 may include one or more packet switched networks, such as the Internet, or any local area networks, wide area networks, enterprise private networks, cellular networks, phone networks, mobile communications networks, or any combination of the above. In one embodiment, devices 112, 114, wearable device 117 and server 102 may be a programmable logic controller or PLC.

Server 102 includes a software engine that delivers applications, data, program code and other information to devices 112, 114 and wearable device 117. The software engine of server 102 may perform other processes such as transferring multimedia data in a stream of packets that are interpreted and rendered by a software application as the packets arrive. FIG. 1 further shows that server 102 includes a database or repository 104, which may be a relational database comprising a Structured Query Language (SQL) database stored in an SQL server or a database that adheres to the noSQL paradigm. Devices 112, 114 and wearable device 117 may also each include databases. The database 104 may serve data, as well as related information, used by server 102 and devices 112, 114 and wearable device 117 during the course of operation of the invention.

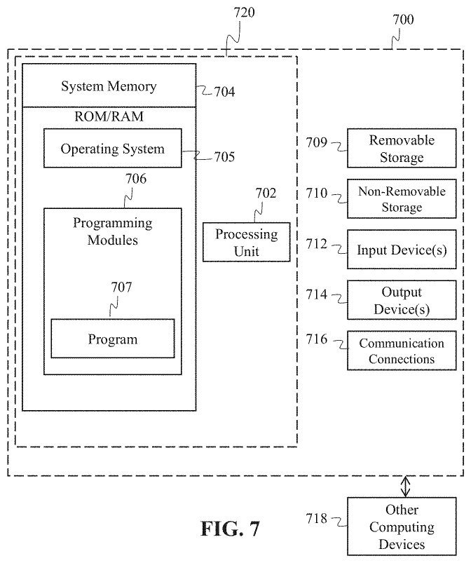

Devices 112, 114 and wearable device 117 and server 102 may each include program logic comprising computer source code, scripting language code or interpreted language code that perform various functions of the disclosed embodiments. In one embodiment, the aforementioned program logic may comprise program module 707 in FIG. 7. It should be noted that although FIG. 1 shows only one device 112, ond device 114, one wearable device 117 and one server 102, the system of the disclosed embodiments support any number of terminals, servers, mobile devices and wearable devices connected via network 106. Also note that although server 102 is shown as a single and independent entity, in one embodiment, server 102 and its functionality can be realized in a centralized fashion in one computer system or in a distributed fashion wherein different elements are spread across several interconnected computer systems.

Various types of data may be stored in the database 104 of server 102 for effectuating a measured change by a user. A measured change by the user may include at least one of a sleep pattern, a fat percentage, a muscle mass change, a change in reading habits, usage of IOT devices, cyber hygiene (computing behavior or practices such as turning off your computer, maintaining records, changing password, etc., reading habits. However, it is understood that the present invention may be designed for effectuating other measured changes.

The database 104 may store in a user record user data, which may include user baseline data of the user, first algorithms, second algorithms, third algorithms, performance indicators, performance indicator user data, user baseline data, unexpected reward messages, loss aversion messages, inquiry messages, user response messages.

User data may also include user identifying data, an email address, physical address, birth data, physical address, medical history and data, user baseline data, performance indicators, performance indicator user data, unexpected reward messages, loss aversion messages, inquiry messages, user response messages. However, it is understood that other information may also be included in user data.

User baseline data may be defined as data received to establish an initial baseline of the user from which a measured change may be measured. User baseline data may include blood glucoses, blood pressures, BMIs, body weight, calories consumed, calories consumed, heart rate, step count, total sleep, mindfulness, reproductive health, basal body temperature, sexual activities, menstrual flow, intermenstrual bleedings, and, ovulation tests, awake time, deep/light/REM sleep, distance, duration, data related number of steps, pages to read, IOT device usage, cyber hygiene etc. It is also understood that the user baseline data may include data provided by user in SMS format and also data provided by other information sources. The information sources may include a wearable device, a third-party device, motion sensor, etc. In one embodiment, the information source may be wearable device 117. It is also understood of other types of data may also be also be stored as user baseline data and are within the spirit and scope of the present invention.

An unexpected expected reward and loss aversion messages are intended to cause a measured change for the user. The unexpected reward message is configured to provide to the user a reward or benefit for accomplishing or satisfying a certain performance indicator. Unexpected reward messages may include digital coupons for new clothes, coupons for book, etc. The unexpected reward messages may also include SMS messages including other types of other types of rewards. Additionally, the unexpected reward messages may also include other types of messages that within the spirit and scope of the present invention. The loss aversion messages are also intended to cause a measured change. In one embodiment, the loss aversion message is transmitted to the user device 112, 114 and is intended to motivate the user to do better. The loss aversion message may be transmitted to the user if a user does not satisfy a certain performance indicator. The loss aversion message may also include digital coupons for new clothes, coupons for book, etc. The unexpected reward messages may also include SMS messages including other types of other types of rewards. Additionally, the unexpected reward messages may also include other types of messages that within the spirit and scope of the present invention.

The first algorithms include logic for facilitating the measured change of a user based upon the baseline data received. Examples of the first algorithms have been included in the Appendix. The first algorithms may be stored in the attached database. The first algorithm may include logic that uses the baseline data to determine a performance indicator for facilitating the measured change. The performance indicator may be determined by the first algorithm for facilitating the measured change. Performance indicators may include a unit for blood glucoses, blood pressures, BMIs, body weight, calories consumed, calories consumed, heart rate, step count, total sleep, mindfulness, reproductive health, basal body temperature, sexual activities, menstrual flow, intermenstrual bleedings, and, ovulation tests, awake time, deep/light/REM sleep, distance, duration, data related number of steps, pages to read, IOT device usage, cyber hygiene etc. The performance indicator may be defined as a threshold amount of change relative to the user baseline data.

Performance indicator user data may be provided by user and may include input into devices 112, 114 or via wearing devices 117 or other information sources or applications. Performance indicator user data may be provided via SMS message from the remote computing devices 112, 114. However, other means of providing the performance indicator user data is contemplated by this invention. The performance indicator user data may be related to blood glucose level of the user, blood pressures, BMIs, body weight, calories consumed, calories consumed, heart rate, step count, total sleep, mindfulness, reproductive health, basal body temperature, sexual activities, menstrual flow, intermenstrual bleedings, and, ovulation tests, awake time, deep/light/REM sleep, distance, duration, data related number of steps, pages to read, IOT device usage, cyber hygiene etc.

The wearable device 117 may include devices that can be worn on the user's person and is formatted to read and store a plurality of data associated with the user within in a certain time period. By way of example and not limitation, such physical activity data may include a number of calories burned, type of physical activity, length of time dedicated to physical activity, intensity of physical activity, amount of step walked, the amount of sleep, the amount of IOT device usage, user's pulse, user blood pressure. Such information may be transmitted via radio frequency or vendor application to a reception module associated with the application.

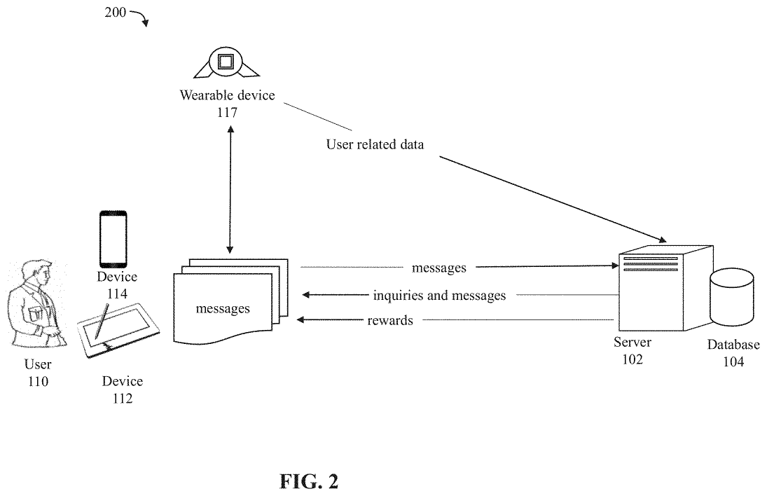

FIG. 2 is a diagram showing the data flow 200 of the apparatus, code, methods, and systems for providing unexpected rewards for a measured change of a user, according to an example embodiment. The data flow show that messages or data may be transmitted between the user 110 devices 112, 114 to the server. Data and information may be input on the graphical user interfaces of remote devices 112, 114 via button push, swipe, gesture, click, entry of SMS text message, etc. Additionally, user related data, such as baseline user data and performance indicator user data may flow from the wearable device 117 to the server and to devices 112 and 114. It is also understood that in addition to wearable devices, other information sources may provide data to the server. In other embodiments, all the apparatus, code, methods and systems may be performed on the devices 112 and 114 without having to transmit data to the server 102.

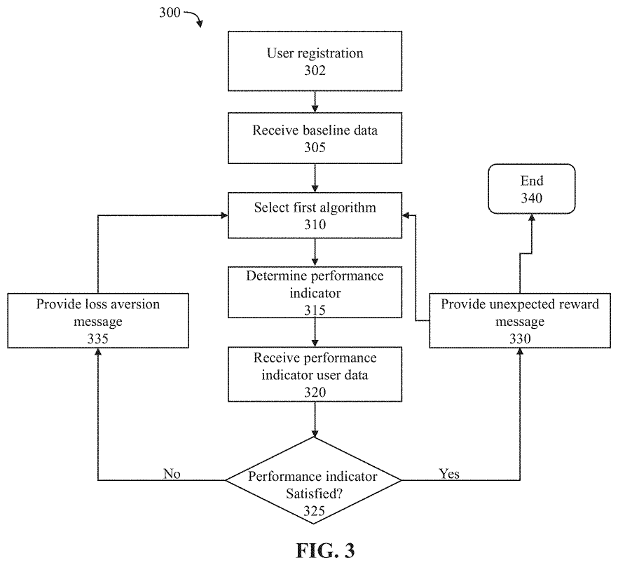

FIG. 3 is a diagram showing a first process flow 300 of the apparatus, code, methods, and systems for providing unexpected rewards for a measured change of a user, according to an example embodiment. In step 302, the user registers with the application. The registration process may be unknown to the user and be performed by a third party, such as an employer, or other third parties that desire a measured change by the user. As mentioned above the measured change may include measured change by the user may include sleep pattern change, a fat percentage, a muscle mass change, a change in reading habits, usage of IOT devices, cyber hygiene (computing behavior or practices such as turning off your computer, maintaining records, changing password, etc.), and reading habits. However, it is understood that the present invention may be designed for effectuating other measured changes.

In step 305, the baseline data of the user is provided. As mentioned above, baseline user data may be provided from a plurality of information sources, such as wearable device 117, or by input from the user via the devices 112, 114. Next the baseline data and other user data is stored in a user record in database 104. Next, in step 310, a first algorithm for facilitating the measured change of a user based upon the baseline data received is selected from the plurality of algorithms stored in an attached database. The system may be preprogrammed to select an algorithm depending on the baseline data received. For example, if the user is determined to be very inactive or have an unhealthy BMI, then the system may select a first algorithm that increases user activity or creates a healthier BMI. In other embodiments, the first algorithm is automatically selected given input from the user.

Next, in step 315, based upon the baseline data and the first algorithm, at least one performance indicator for facilitating the measured change is calculated and stored in the corresponding user record. Next, in step 320, after a predetermined amount of time (which is calculated by the first algorithm), over the communications network, a first set of performance indicator user data is received from the information sources associated with the user. As mentioned above, the information source may be the wearable device 117 from input received from the rThe performance indicator user data is associated with the user baseline data and the performance indicator. Using the example above where the first algorithm is for increasing user activity or creating a healthier BMI, the performance indicator user data may include a threshold number of steps the user should take over a period of time and the user performance indicator user data is the amount of steps the user takes over a period of time based upon the information provided by the information sources. Next, in step 325, a determination is made, based upon the first set of performance indicator user data received, if the performance indicator for the measured change was satisfied. For example, if the performance indicator was for the user to walk more than 10,000 steps over a period of time and if the user walked 11,000 steps over the period of time, then the performance indicator would be satisfied, and the process would move to step 330. On the other hand, if the performance indicator is not satisfied, then the process would move to step 335. In step 330, an unexpected reward message would be provided to the remote computing device 112, 114 based upon the fact that the user satisfied the performance indicator. In step 335, a loss aversion message would be provided to the remote computing device 112, 114 based upon the fact that the user did not satisfy the performance indicator. Next, after either the unexpected reward message and reward message has been provided, steps 310 through 325 are repeated. By repeating step 310 through 325, the apparatus, code, system and methods are configured to recalculate the objective based upon the information received. In other embodiments, if the measured change has been satisfied, then the system may move to step 340 to end the process for a predetermined amount of time.

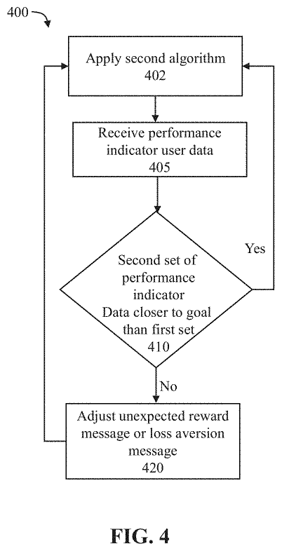

FIG. 4 is a diagram showing a second process flow 400 of the apparatus, code, methods, and systems for providing unexpected rewards for a measured change of a user, according to an example embodiment. FIG. 4 illustrates how the system can determine the effectiveness of the unexpected reward messages and the loss aversion messages. In step, 402, the system will first apply a second algorithm. Next in step 405, sets of performance indicator user data is received (at least a first set and a second set of performance indicator user data). Next, in step 410, the system determines if the second set of performance indicator user data received is at least closer to the performance indicator or goal than the first set of performance indicator user data received. This process allows the system to determine if the reward messages and loss aversion messages were helpful or should be calculated. If the second set of performance indicator user data received is not closer to the performance indicator or goal than the first set of performance indicator user data received, then the system moves to step 420 and the loss aversion messages or unexpected reward messages are updated to another message. The messages may be updated by content of message, reward offered, demographic, timing of message, frequency of message etc.

On the other hand, in step 410, if the second set of performance indicator user data received is closer to the performance indicator or goal than the first set of performance indicator user data received, then the system moves back to 402 to continually apply the second algorithm to adjust the efficacy of the loss aversion messages and unexpected reward messages sent to the users.

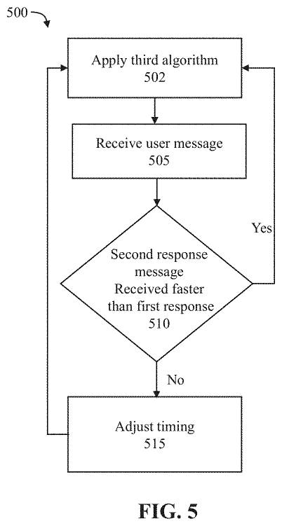

FIG. 5 is a diagram showing a third process flow 500 of the apparatus, code, methods, and systems for providing unexpected rewards for a measured change of a user, according to an example embodiment. FIG. 5 illustrates that the invention is configured providing messages that adjust when the to send messages to the user. The invention uses a third algorithm, stored in the attached database, to determine the second efficacy of the predetermined amount of times for inquiry sending messages to the user. In step 505, the system is configured for receiving the messages and performance indicator user data from the user (at least a first message and a second message from the user). Next, in step 510 the system determines the efficacy of the predetermined amount of time for sending inquiry messages to the user. If the user response is faster when the invention sends an inquiry message to the user at a first time of day (i.e., in the morning) than when an inquiry message is sent to the user at a second time of day (i.e., at evening time), then the invention will adjust when the inquiries will be sent to the user to send the messages so that response messages are received faster (see step 515). On the other hand, if the user responses are faster when inquires to the user at a second time of day (i.e., at evening time), then, the invention will reapply the algorithm to determine an even more precise time as to the best time to send messages so that the amount of engagement by the user is maximized. Additionally, the system may be configured to determine the best time to send messages by taking into account into account special events, wherein special events include birthday, holidays, days off, weekends, vacation days etc. Additionally, the invention is configured that if user messages are not received within a second predetermined amount of time, then code is configured for sending an engaging message for reinitiating correspondence with the user.

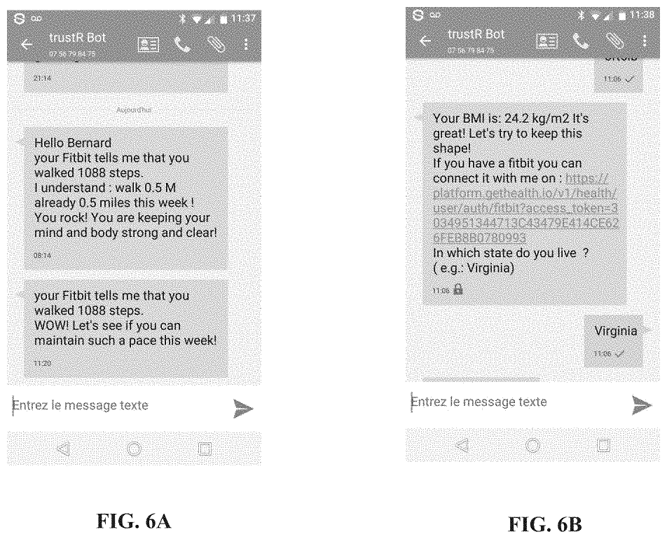

As mentioned above, the baseline data and performance indicator user data in one embodiment may be provided by user messages, via SMS message, from the remote computing device. FIG. 6A is a diagram illustrating a first graphical display 600 used with the apparatus, code, methods, and systems for providing unexpected rewards for a measured change of a user, according to an example embodiment and FIG. 6B is a diagram illustrating a second graphical display 650 used with the apparatus, code, methods, and systems for providing unexpected rewards for a measured change of a user, according to an example embodiment.

FIGS. 6A and 6B illustrate displays wherein the user may transit the baseline data and performance indicator user data via SMS message. FIG. 6A also illustrates that the invention may user the wearable device 117 or a fit bit that provide both baseline data and performance indicator user data.

FIG. 7 is a block diagram of a system including an example computing device that be used by devices 112, 114, wearable device 117, and server 102. Consistent with the embodiments described herein, the aforementioned actions may be implemented in a computing device, such as the computing device 700 of FIG. 9. Any suitable combination of hardware, software, or firmware may be used to implement the computing device 700. The aforementioned system, device, and processors are examples and other systems, devices, and processors may comprise the aforementioned computing device. Furthermore, computing device 700 may comprise an operating environment for devices and systems, as described above. Data flow, 200 and processes 300, 400, and 500, may operate in other environments and are not limited to computing device 700.

With reference to FIG. 7, a system consistent with an embodiment of the invention may include a plurality of computing devices, such as computing device 700. In a basic configuration, computing device 700 may include at least one processing unit 702 and a system memory 704. Depending on the configuration and type of computing device, system memory 704 may comprise, but is not limited to, volatile (e.g. random access memory (RAM)), non-volatile (e.g. read-only memory (ROM)), flash memory, or any combination or memory. System memory 704 may include operating system 705, and one or more programming modules 706. Operating system 705, for example, may be suitable for controlling computing device 700's operation. In one embodiment, programming modules 706 may include, for example, a program module 707 for executing the actions of the device. Furthermore, embodiments of the invention may be practiced in conjunction with a graphics library, other operating systems, or any other application program and is not limited to any particular application or system. This basic configuration is illustrated in FIG. 7 by those components within a dashed line 720.

Computing device 700 may have additional features or functionality. For example, computing device 700 may also include additional data storage devices (removable and/or non-removable) such as, for example, magnetic disks, optical disks, or tape. Such additional storage is illustrated in FIG. 7 by a removable storage 709 and a non-removable storage 710. Computer storage media may include volatile and nonvolatile, removable and non-removable media implemented in any method or technology for storage of information, such as computer readable instructions, data structures, program modules, or other data. System memory 704, removable storage 709, and non-removable storage 710 are all computer storage media examples (i.e. memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 700. Any such computer storage media may be part of device 700. Computing device 700 may also have input device(s) 712 such as a keyboard, a mouse, a pen, a sound input device, a camera, a touch input device, etc. Output device(s) 714 such as a display, speakers, a printer, etc. may also be included. The aforementioned devices are only examples, and other devices may be added or substituted.

Computing device 700 may also contain a communication connection 716 that may allow device 700 to communicate with other computing devices 718, such as over a network in a distributed computing environment, for example, an intranet or the Internet. Communication connection 716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 704, including operating system 705. While executing on processing unit 702, programming modules 706 (e.g. program module 707) may perform processes including, for example, one or more of the stages of the data flow, 200 and processes 300, 400, and 500 and for providing interfaces 600 and 650 as described above. The aforementioned processes are examples, and processing unit 702 may perform other processes. Other programming modules that may be used in accordance with embodiments of the present invention may include electronic mail and contacts applications, word processing applications, spreadsheet applications, database applications, slide presentation applications, drawing or computer-aided application programs, etc.

According to further embodiments of the present disclosure, the system described herein correlates multiple subjective and objective measures to provide improved accuracy and ground truth relating to a patient's actual health status, activity level, or drug adherence. In one such embodiment, the system described herein initially uses a learning AI to capture language/speech patterns including for instance phrasings, word choice, sentiment, and tone that a user employs in written communication and has demonstrated themselves to be responsive to. The user's own speech patterns are then used to create a group of Target Speech Patterns. The system then tests these Target Speech Patterns against the user via sms or web interface to confirm phrasings, word choice, sentiment, and tone that provide the best response rates and sentiment.

Next, the system adapts standardized questionnaire forms, including for instance SF36, SF12, EQ5D, and QDIS known in the medical arts into tone, word choice, syntax, and timing to which a user has demonstrated heightened responsiveness to generate a customized Interview Script. The Interview Script is then administered to the user via asynchronous conversation, including for instance via a web chat or SMS in order to ascertain health status, with the results of that administration being converted back into the standardized response model for the given survey.

The program monitors speech patterns for changes in speech, word choice, phrasing, sentiment or tone from a user's customary manner of communicating. In case of an unexpected change, the system begins a thread of inquiry to determine if there is anything changed about a user's well-being or health status, including for instance using a standardized health questionnaire or targeted health status questions chosen to elicit responses related to complications associated with the user's known diseases or injuries.

According to further embodiments of the present disclosure, physical activity observed by for instance, a motion tracking sensor is normalized for the types of walking activity. For instance, in a setting where a user lives in multi-story housing and needs to climb stairs, as compared to a user who lives in a rural setting and may have uneven ground to walk over regularly, as compared to a resident in an urban setting who has to traverse greater portions of pavement, the system normalizes said data to observe and capture relative improvements that may or may not result from interventions such as change in drug regimens, therapy or exercise programs, diet, etc. such that data from all of those various classes of patients may be compared with or distinguished from one another.

According to further embodiments of the present disclosure, the system tailors the subject matter of standardized health assessments including for instance “difficulty walking up stairs” as part of the “mobility” portion of the SF36, but replacing “stairs” with features or topography that the system has learned the user is likely to encounter in their own experience. Afterward, when said data is collected from a large enough sample, the software standardizes said data to be interoperable with the validated construct.

Similarly, answers to specific questions may be additionally validated by external sensors. For instance, self-reported outcomes and progress such as activity levels may be validated using accelerometer readings from within a cellular phone or location history from a phone's GPS. Self-reported dietary progress for diabetics may be checked occasionally against blood tests or continuous glucose meters in order to validate the present self-reported data and similarly weigh and adjust future self-reporting by the user. Refill statistics for a given medication may be correlated with patient-provided answers to adherence questions to weigh future patient responses.

The outputs of these measures may then be communicated to the user as well as their care providers or anonymized for provision to interested 3^{rd} parties such as pharmaceutical companies or insurance companies. In the case of the care providers, the system is capable of organizing outputs and results based on varying levels of granularity. At one instance, the system is capable of showing the provider the real health status for a specific patient. In a second instance, the system is capable of showing the provider any one particular outcome across their patients that have been treated in a with a particular intervention or medication, or based on demographic or psychographic criteria known in the medical arts.

Generally, consistent with embodiments of the invention, program modules may include routines, programs, components, data structures, and other types of structures that may perform particular tasks or that may implement particular abstract data types. Moreover, embodiments of the invention may be practiced with other computer system configurations, including hand-held devices, multiprocessor systems, microprocessor-based or programmable consumer electronics, minicomputers, mainframe computers, and the like. Embodiments of the invention may also be practiced in distributed computing environments where tasks are performed by remote processing devices that are linked through a communications network. In a distributed computing environment, program modules may be located in both local and remote memory storage devices.

Furthermore, embodiments of the invention may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments of the invention may also be practiced using other technologies capable of performing logical operations such as, for example, AND, OR, and NOT, including but not limited to mechanical, optical, fluidic, and quantum technologies. In addition, embodiments of the invention may be practiced within a general purpose computer or in any other circuits or systems.

Embodiments of the present invention, for example, are described above with reference to block diagrams and/or operational illustrations of methods, systems, and computer program products according to embodiments of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the invention have been described, other embodiments may exist. Furthermore, although embodiments of the present invention have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the invention.

A such as although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A computer readable medium storing computer readable program code which when executed on a server is configured for providing unexpected rewards for a measured change of a user, wherein the code is configured such that the information sources include a % wearable device, a third-party device, motion sensor, etc. wherein the code is configured for:
a. providing, over a communications network, to a remote computing device a graphical user interface configured for receiving a plurality of user data and storing, in the attached database, the user data in a plurality of user records;
b. receiving, over the communications network, from a plurality of information sources associated with the user, a plurality of baseline data for the user and storing the baseline data in the attached database;
c. selecting from a plurality of algorithms stored in an attached database, a first algorithm for facilitating the measured change of a user based upon the baseline data received;
d. determining, based upon the baseline data and the first algorithm, at least one performance indicator for facilitating the measured change, and storing the performance indicator in the corresponding user record;
e. receiving, over the communications network, from at least one of the plurality of information sources associated a first set of performance indicator user data;
f. receiving, over the communications network, from asynchronous text-based communication with the use information a second set of performance indicator user data relating to the subject matter of (e);
g. combining the data of the first and second set to create a set of validated data
h. determining based upon the validated data received if the performance indicator for the measured change was satisfied;
i. providing, over the communications network, to the remote computing at least one of an unexpected reward message, and the loss aversion message based upon if the performance indicator for the measured change was satisfied; and,
j. repeating steps c-g until the measured change is satisfied.

2. The computer readable medium storing computer readable program code of claim 1, wherein the code is further configured for:
a. applying a second algorithm, stored in the attached database, to determine a first efficacy of the unexpected reward message and the loss aversion message;
b. adjusting at least one of the unexpected reward message and the loss aversion message such that a second set of performance indicator user data received is at least closer to the performance indicator than the first set of performance indicator user data received; and,
c. repeating steps a-b until the desired changed is satisfied.

3. The computer readable medium storing computer readable program code of claim 2, wherein the code is further configured for
a. applying a third algorithm, stored in the attached database, to determine the second efficacy of the predetermined amount of times for inquiry sending messages to the user;
b. adjusting the predetermined amount of times for sending inquiry messages to the user such that user messages are received within at least in the same amount of or less than the amount of time than the previous user message was received; and,
c) repeating steps a-b until the measured change is satisfied.

4. The computer readable medium storing computer readable program code of claim 1, wherein the plurality of information sources are configured for providing at least one of the following related to the user: blood glucoses; blood pressures; BMIs; body weight; calories consumed; heart rate; step count; movement or general activity levels; total sleep; mindfulness; reproductive health; basal body temperature; sexual activities; menstrual flow; intermenstrual bleedings; and, ovulation tests, awake time, deep/light/REM sleep, distance, duration etc.

5. The computer readable medium storing computer readable program code of claim 1, wherein the unexpected reward messages comprise coupon for new clothes, coupon for book, gift cards having varying monetary values, positive affirmations or emoticon(s) which hold emotional but no monetary value, etc.

6. The computer readable medium storing computer readable program code of claim 1, wherein the measured change comprises at least one of a sleep pattern, a fat percentage, a muscle mass change, a change in reading habits, adherence to exercise regimens, adherence to therapy regimens, adherence to medications, an average respiratory change, or any other sustained change in behavior or biological variable captured by the system.

7. The computer readable medium storing computer readable program code of claim 1, wherein the system captures differences in the nature and frequency of patient daily experiences and then leverages that heterogeneity to improve the fidelity of health outcome measures.

8. A computer readable medium storing computer readable program code which when executed on a server is configured for providing unexpected rewards for a measured change of a user, wherein the code is configured such that the information sources include a wearable device, a third-party device, motion sensor, etc wherein the code is configured for:
a. providing, over a communications network, to a remote computing device a graphical user interface configured for receiving a plurality of user data and storing, in the attached database, the user data in a plurality of user records;
b. receiving, over the communications network, from a plurality of information sources associated with the user, a plurality of baseline data for the user and storing the baseline data in the attached database;
c. initiating conversational asynchronous text communication with a user;
d. monitoring the communications from (c) to determine a user's normal use of at least one of speech patterns, phrasings, word choice, sentiment, or tone;
e. storing the results from (d) in a record associated with the user containing preferred rules for speech patterns, phrasings, word choice, sentiment or tone;
f. maintaining a second database having a standardized medical interview form or survey;
g. adapting the language used in the second database to create a personalized set of linguistic rules for engaging with the user when executing an interview form or survey;
e. recording the results of (g) in a standardized format compatible with that of the original survey or form
f. providing rewards, incentives, or recognition based on a user's meeting specific metrics within the standardized questionnaires, with the timing and value of the rewards being modulated to be unexpected for the user.
